(19) United States
(12) Patent Application Publication
(10) Pub. No.: US 2025/0352767 A1
(43) Pub. Date: Nov. 20, 2025

(54) CATHETER SECUREMENT DEVICES

(71) Applicant: Becton, Dickinson and Company, Franklin Lakes, NJ (US)

(72) Inventors: Ryan Wade, Atlanta, GA (US); Daniel Blanchard, Bountiful, UT (US); Chad Alan Tagge, Sandy, UT (US); Andrew Richards, Durham, NC (US); Christopher Quach, North Salt Lake, UT (US); Weston F. Harding, Lehi, UT (US); Bradley M. Wilkinson, North Haledon, NJ (US)

(21) Appl. No.: 19/207,108

(22) Filed: May 13, 2025

Related U.S. Application Data
(60) Provisional application No. 63/648,824, filed on May 17, 2024.

(51) Int. Cl. A61M 25/02

(52) U.S. Cl. CPC A61M 25/02; A61M 2025/0213; A61M 2025/0266

(57) ABSTRACT

A securement device for securing a catheter assembly may include one or more anchor pads having an adhesive surface. The securement device may include a retainer positioned on the one or more anchor pads. The retainer may include one or more retaining edges configured to contact a wing of the catheter assembly. The retainer may include a catheter angle adjuster. The securement device may include an extension tube securement element disposed on the one or more anchor pads. Another securement device for securing a catheter assembly may include a bracelet configured to wrap around a wrist of a patient. Another securement device for securing a catheter assembly may include a retainer having a bottom cover and a top cover hingedly coupled to the bottom cover. When the top cover is in the closed position, the retainer may secure the catheter assembly between the top cover and the bottom cover.

### RELATED APPLICATIONS

This application claims the benefit of U.S. Provisional Patent Application No. 63/648,824, which was filed on May 17, 2024, which is incorporated herein in its entirety.

### BACKGROUND

A common type of catheter assembly includes a catheter that is over-the-needle. As its name implies, the catheter that is over-the-needle may be mounted over an introducer needle having a sharp distal tip. The catheter assembly may include a catheter adapter, the catheter extending distally from the catheter adapter, and the introducer needle extending through the catheter. The catheter and the introducer needle may be assembled such that the distal tip of the introducer needle extends beyond the distal tip of the catheter with a bevel of the introducer needle facing up away from skin of the patient immediately prior to insertion into the skin. The catheter and the introducer needle are generally inserted at a shallow angle through the skin into a blood vessel of the patient.

In order to verify proper placement of the introducer needle and/or the catheter in the blood vessel, a clinician may confirm that there is flashback of blood in a flashback chamber of the catheter assembly or in a space between the introducer needle and the catheter. Once placement of the introducer needle within the blood vessel has been confirmed, the clinician may remove the introducer needle, leaving the catheter in place in the blood vessel for future blood withdrawal or fluid infusion.

In order to keep the catheter properly positioned for a duration of a patient's treatment, the catheter assembly may be secured to the patient. Securing the catheter assembly to the patient may reduce a likelihood of kinking or dislodgement of the catheter. The catheter assembly is frequently secured to the patient using an adhesive or sutures. Suturing increases the time to secure the catheter assembly to the patient and may also increase a risk of infection. Current adhesives do not do enough to prevent rotation of the catheter or allow adjustment of an angle of the catheter entering the skin. Also, current catheter assembly securement methods do not manage an extension tube of the catheter assembly.

The subject matter claimed the present disclosure is not limited to embodiments that solve any disadvantages or that operate only in environments such as those described above. Rather, this background is only provided to illustrate one example technology area where some implementations described the present disclosure may be practiced.

### SUMMARY

The present disclosure relates generally to securement devices for securing a catheter assembly, as well as related systems and methods. According to a first set of embodiments, a securement device may include one or more anchor pads, which may include an adhesive surface disposed on an underside thereof and that is configured to attach to a skin surface of a patient. In some embodiments, the securement device may include a retainer positioned on an upper surface of the one or more anchor pads.

In some embodiments, the retainer may include a first retaining edge configured to contact a top of a first wing of the catheter assembly. In some embodiments, the retainer may include a second retaining edge configured to contact a top of a second wing of the catheter assembly opposite the first wing. In some embodiments, the retainer may include a catheter angle adjuster. In some embodiments, the catheter angle adjuster may include a nose portion configured to deflect a strain relief of the catheter assembly at a predetermined angle.

In some embodiments, the retainer may include a first proximal leg and a second proximal leg. In some embodiments, the first proximal leg and the second proximal leg extend downward from a central portion. In some embodiments, the retainer may include a first distal leg and a second distal leg. In some embodiments, the first distal leg and the second distal leg may extend downward from the central portion. In some embodiments, the first proximal leg and the first distal leg may be on an opposite side of a midline as the second proximal leg and the second distal leg.

In some embodiments, the first retaining edge may extend between the first proximal leg and the first distal leg. In some embodiments, the second retaining edge may extend between the second proximal leg and the second distal leg. In some embodiments, the first proximal leg and the first distal leg may be configured to inhibit longitudinal movement of the first wing. In some embodiments, the second proximal leg and the second distal leg may be configured to inhibit longitudinal movement of the second wing.

In some embodiments, the securement device may include an extension tube securement element disposed on the upper surface of the one or more anchor pads. In some embodiments, the extension tube securement element may include a track, and an extension tube of the catheter assembly may be configured to be press fit within the track. In some embodiments, the track may be U-shaped and configured to turn the extension tube 180 degrees.

In some embodiments, the one or more anchor pads may include a first anchor pad and a second anchor pad laterally offset from the midline. In some embodiments, the retainer may be positioned on the upper surface of each of the first anchor pad and the second anchor pad. In some embodiments, the first anchor pad and the second anchor pad may be shaped so as to allow ingress and egress of a portion of the catheter assembly therebetween.

According to a second set of embodiments, a securement device for securing a catheter assembly may include a bracelet configured to wrap around a wrist of a patient. In some embodiments, the securement device may include a retainer positioned on an upper surface of the bracelet. In some embodiments, the retainer may include a first retaining edge configured to contact a top of a first wing of the catheter assembly. In some embodiments, the retainer may include a second retaining edge configured to contact a top of a second wing of the catheter assembly opposite the first wing. In some embodiments, the retainer may include a nose portion configured to deflect a strain relief of the catheter assembly at a predetermined angle or a wedge-shaped platform configured to support a catheter adapter of the catheter assembly.

In some embodiments, the securement device may include an extension tube securement element disposed on the upper surface of the bracelet. In some embodiments, the securement device may include a rigid support. In some embodiments, the rigid support may include a first end coupled to the bracelet and configured to extend from the bracelet toward fingers of the patient. In some embodiments, the securement device may include a band coupled to a second end of the rigid support. In some embodiments, the band may be configured to wrap around the fingers.

According to another set of embodiments, a securement device for securing a catheter assembly may include one or more anchor pads, which may include an adhesive surface disposed on an underside thereof and that is configured to attach to a skin surface of a patient. In some embodiments, the securement device may include a retainer, which may include a bottom cover fixed with respect to an upper surface of the one or more anchor pads. In some embodiments, the bottom cover may include a latch feature.

In some embodiments, the securement device may include a top cover hingedly coupled to an upper surface of the bottom cover. In some embodiments, the top cover may include a distal end, which may include a nose portion configured to deflect a strain relief of the catheter assembly at a predetermined angle. In some embodiments, the top cover may include a corresponding latch feature. In some embodiments, in response to the top cover moving from an open position to a closed position, the latch feature and corresponding latch feature are secured together. In some embodiments, when the top cover is in the closed position, the retainer is configured to secure the catheter assembly between the top cover and the bottom cover.

In some embodiments, the top cover may be configured to contact an upper surface of a wing of the catheter assembly when the top cover is in the closed position to inhibit transverse movement of the catheter assembly. In some embodiments, the latch feature may be configured to contact a distalmost surface of a wing of the catheter assembly when the top cover is in the closed position to inhibit distal movement of the catheter assembly. In some embodiments, the top cover may include a gap disposed between the corresponding latch feature and the proximal end to receive a side port of the catheter adapter and at least a portion of a wing of the catheter assembly.

In some embodiments, the top cover includes a proximal end hingedly coupled to the upper surface of the bottom cover. In some embodiments, the corresponding latch feature may be disposed between the proximal end and the distal end. In some embodiments, the top cover may be aligned with a midline in response to the top cover being in the open position and the closed position.

In some embodiments, the top cover may include a side, which may include one or more hinges coupled to the upper surface of the bottom cover. In some embodiments, the corresponding latch feature may be disposed on an opposite side of the midline than the one or more hinges.

In some embodiments, the proximal end of the top cover may include one or more hinges coupled to the upper surface of the bottom cover. In some embodiments, the bottom cover may include multiple latch features. In some embodiments, the distal end of the top cover may include multiple corresponding latch features configured to couple with multiple latch features in the closed position.

In some embodiments, the side of the top cover may include a distal hinge and a proximal hinge. In these embodiments, the bottom cover may include multiple latch features. In some embodiments, an opposite side as the side of the top cover may include multiple corresponding latch features configured to couple with the multiple latch features in the closed position.

In some embodiments, the bottom cover may be coupled to a wedge-shaped platform configured to support the catheter assembly at a predetermined angle. In some embodiments, the bottom cover may be coupled to a rotatable wedge. In some embodiments, the rotatable wedge may include a first platform at a first height and a second platform at a second height greater than the first height. In some embodiments, the retainer may include a pin extending through the rotatable wedge to the bottom cover, and the rotatable wedge may rotate around the pin. In some embodiments, the first platform and the second platform may be angled downwardly away from the pin and towards an outer edge of the rotatable wedge.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present disclosure. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is an upper perspective view of an example securement device, according to some embodiments;

FIG. 1B is a top view of the securement device of FIG. 1A, according to some embodiments;

FIG. 1C is an exploded view of the securement device of FIG. 1A, according to some embodiments;

FIG. 1D is an upper perspective view of example anchor pads of the securement device of FIG. 1A, according to some embodiments;

FIG. 1E is a top view of an example retainer of the securement device of FIG. 1A, according to some embodiments;

FIG. 1F is an upper perspective view of the securement device of FIG. 1A coupled to an example catheter assembly, according to some embodiments;

FIG. 1G is a top view of the securement device of FIG. 1A coupled to the example catheter assembly, according to some embodiments;

FIG. 1H is a rear view of the retainer of FIG. 1E, according to some embodiments;

FIG. 1I is a side view of the retainer of FIG. 1E, according to some embodiments;

FIG. 2A is an upper perspective view of an example anchor pad and an example track for securing an extension tube, according to some embodiments;

FIG. 2B is a cross-sectional view of the track, according to some embodiments;

FIG. 2C is an upper perspective view of another example securement device including the track and coupled to the catheter assembly, according to some embodiments;

FIG. 3A is an upper perspective view of another example securement device having a bracelet, according to some embodiments;

FIG. 3B is an upper perspective view of the securement device of FIG. 3A coupled to a patient, according to some embodiments;

FIG. 3C is a side view of the securement device of FIG. 3A coupled to the patient, according to some embodiments;

FIG. 4A is a side view of another example securement device, according to some embodiments;

FIG. 4B is a top view of the securement device of FIG. 4A, according to some embodiments;

FIG. 4C is a top view of a variation of the securement device of FIG. 4A, according to some embodiments;

FIG. 4D is a side view of another variation of the securement device of FIG. 4A having an example wedge-shaped platform, according to some embodiments;

FIG. 5A is a cross-sectional view of an example rotatable wedge in a first position, according to some embodiments;

FIG. 5B is a cross-sectional view of the rotatable wedge in a second position, according to some embodiments; and

FIG. 5C is a top view of the rotatable wedge, according to some embodiments.

### DESCRIPTION OF EMBODIMENTS

Regarding terms used in the present disclosure, it should be understood the terms are for the purpose of describing some particular embodiments, and the terms do not limit the scope of the concepts provided in the present disclosure. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Singular forms of “a,” “an,” and “the” include plural references unless the context clearly dictates otherwise. Also, the words “including,” “has,” and “having,” as used in the present disclosure, including the claims, shall have the same meaning as the word “comprising.”

As used in the present disclosure, the term “proximal” refers to a location with respect to a device during normal use that is closest to the clinician and farthest from the patient. With respect to “proximal,” a “proximal portion” or a “proximal end portion” of, for example, a catheter disclosed in the present disclosure includes a portion of the catheter intended to be near a clinician when the catheter is used on a patient. A “proximal end” of, for example, the catheter includes an end of the catheter intended to be near the clinician when the catheter is used on the patient.

The term “distal” refers to a location with respect to a device during normal use that is farthest from the clinician and closest to the patient. With respect to “distal,” a “distal portion” or a “distal end portion” of, for example, a catheter disclosed in the present disclosure includes a portion of the catheter intended to be near or in a patient when the catheter is used on the patient. A “distal end” of, for example, the catheter includes an end of the catheter intended to be near or in the patient when the catheter is used on the patient.

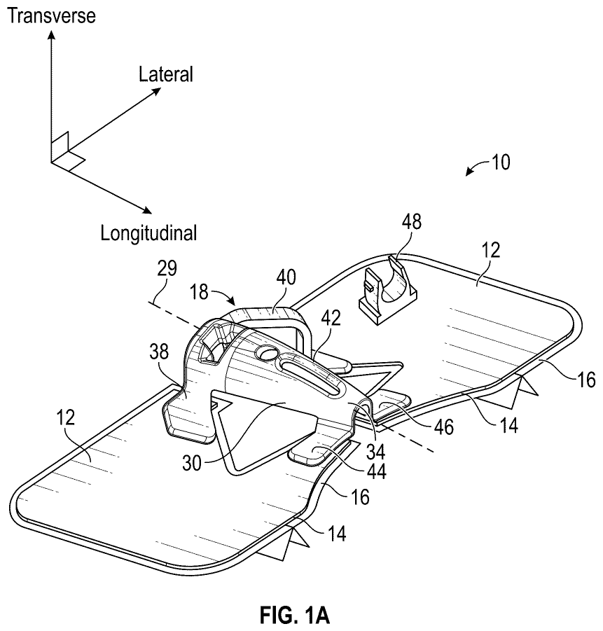

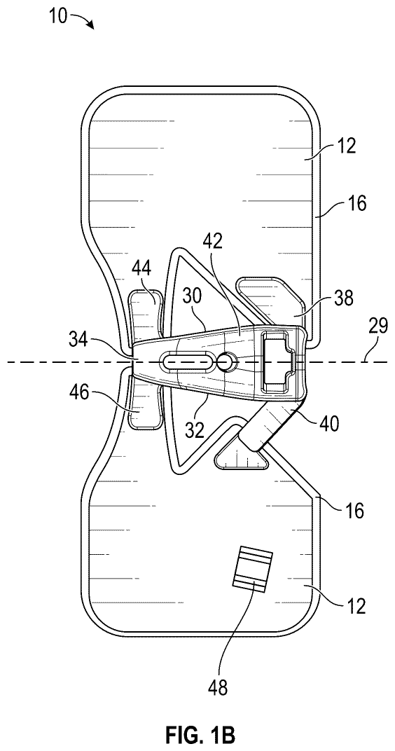

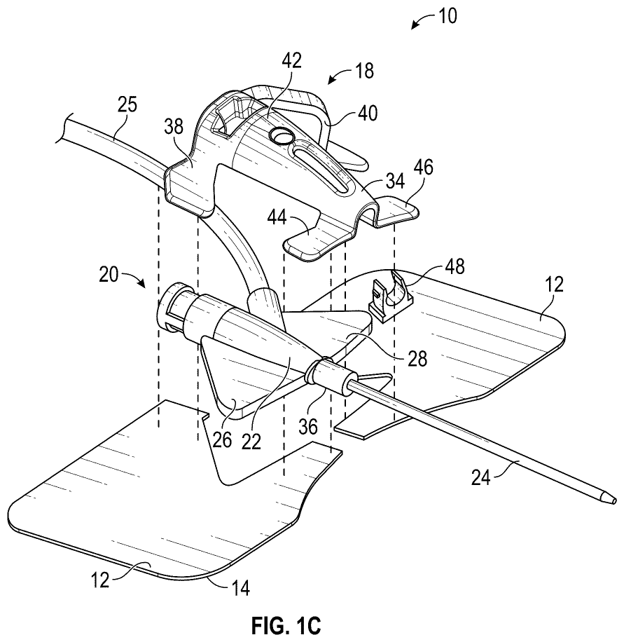

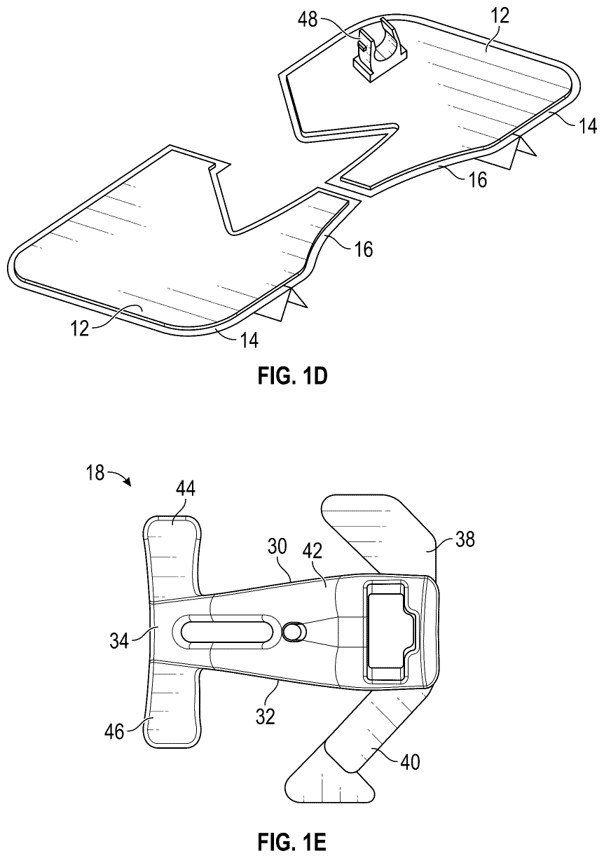

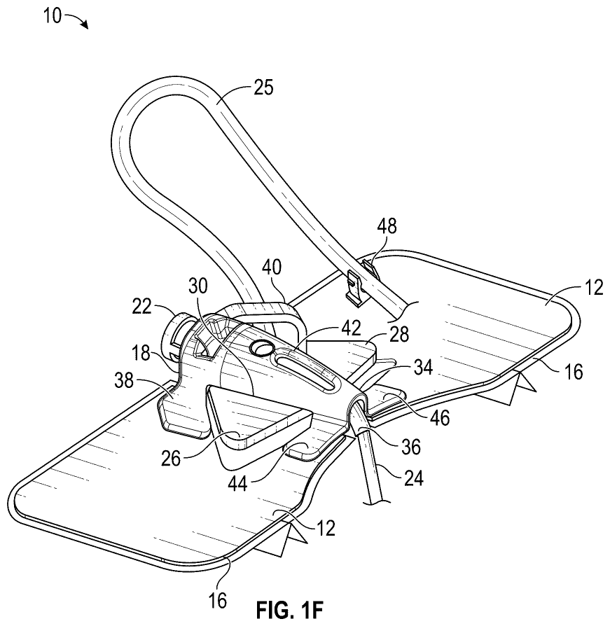

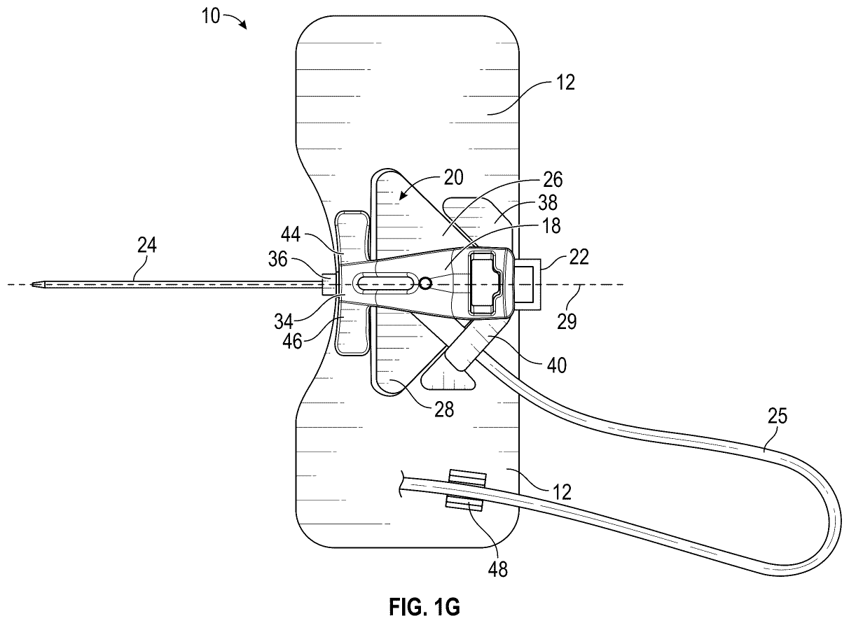

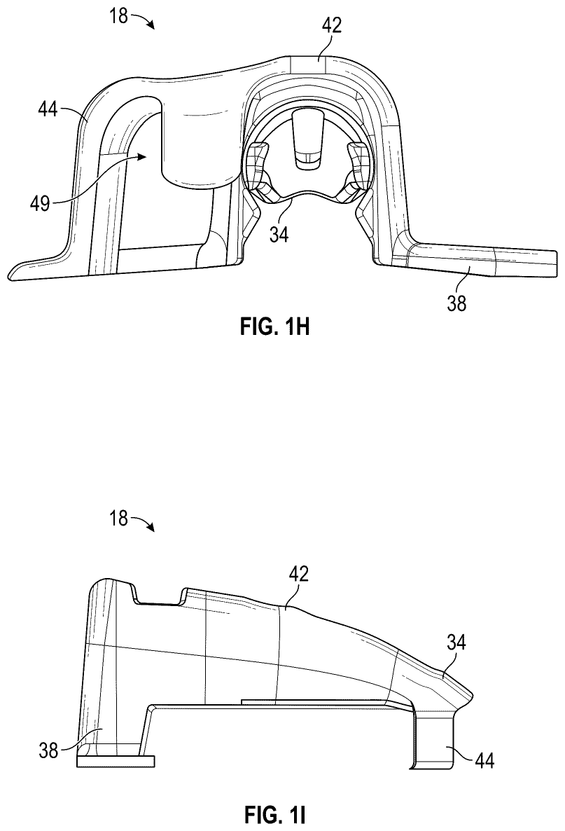

To assist in the description of a device, the following coordinate terms may be used (for example, see FIG. 1A). A “longitudinal axis” is generally parallel to the axis of a channel of the retainer, through which the catheter assembly extends. A “lateral axis” is normal to the longitudinal axis. A “transverse axis” extends normal to both the longitudinal and lateral axes. In addition, as used in the present disclosure, “the longitudinal direction” refers to a direction substantially parallel to the longitudinal axis; “the lateral direction” refers to a direction substantially parallel to the lateral axis; and “the transverse direction” refers to a direction substantially parallel to the transverse axis.

Referring now to FIGS. 1A-1H, a securement device 10 may include one or more anchor pads 12. In some embodiments, the one or more anchor pads 12 may include an adhesive surface 14 disposed on an underside thereof and that is configured to attach to a skin surface of a patient. In some embodiments, one or more liners 16 may cover the adhesive surface 14 prior to adhering the adhesive surface 14 to the patient.

In some embodiments, the securement device 10 may include a retainer 18 positioned on an upper surface of the one or more anchor pads 12. As illustrated, for example, in FIG. 1C, a catheter assembly 20 may be secured and stabilized within the retainer 18. In some embodiments, the catheter assembly 20 may include an arterial catheter, a midline catheter, a dialysis catheter, a Central Venous Catheter (“CVC”), a Peripherally Inserted Central Catheter (“PICC”), a Foley catheter, a urinary catheter, a feeding tube, a balloon catheter, or another suitable catheter.

In some embodiments, the catheter assembly 20 may include a catheter adapter 22, which may include a distal end and a proximal end. In some embodiments, the catheter assembly 20 may include a catheter 24 extending from the distal end of the catheter adapter 22. In some embodiments, the catheter adapter 22 may include a side port and an extension tube 25 extending from the side port. In some embodiments, the catheter assembly 20 may include one or more wings extending outwardly from the catheter adapter 22. In some embodiments, the catheter assembly 20 may include a first wing 26 opposite a second wing 28.

As illustrated, for example, in FIG. 1A, the one or more anchor pads 12 may include a first anchor pad and a second anchor pad which may each include a cut out on their interior edges to permit passage of portions of the catheter assembly 20 such as the wings, which may be flexible. In some embodiments, the first anchor pad and the second anchor pad may be shaped so as to allow ingress and egress of the portions of the catheter assembly 20 therebetween. In some embodiments, the first anchor pad and the second anchor pad may be laterally offset from a midline 29.

In some embodiments, the retainer 18 may include a first retaining edge 30 configured to contact a top of the first wing 26. In some embodiments, the retainer 18 may include a second retaining edge 32 configured to contact a top of the second wing 28. In some embodiments, the first retaining edge 30 and the second retaining edge 32 contacting the first wing 26 and the second wing 28, respectively, prevents rocking, lateral movement, or cantilevering of the catheter 24.

In some embodiments, the retainer 18 may include a catheter angle adjuster. In some embodiments, the catheter angle adjuster may include a nose portion 34 configured to deflect a strain relief 36 of the catheter assembly 20 at a predetermined angle. In some embodiments, an angle of the nose portion 34 allows the catheter adapter 22 to be positioned substantially parallel to the skin surface, which may provide a lower profile and prevent the catheter adapter 22 from protruding from the skin surface and catching on items, such as bandages.

In some embodiments, the strain relief 36 may be disposed at the distal end of the catheter adapter 22 and may support the catheter 24. In some embodiments, the strain relief 36 may be constructed of a flexible material. In some embodiments, the strain relief 36 may be more flexible than the proximal end of the catheter adapter 22. In some embodiments, the nose portion 34 may be angled downward and/or configured to impinge against an antimicrobial disc (not illustrated) to retain the antimicrobial disc in an interference fit between the nose portion 34 and the skin surface. U.S. Patent App. Pub. No. 2020/0330733, filed Apr. 16, 2020, entitled “CATHETER SECUREMENT DEVICE INCLUDING EXTENDED ANCHOR PAD AND RELEASE LINER CLASPING FEATURES,” is hereby incorporated by reference in its entirety.

In some embodiments, the retainer 18 may include a first proximal leg 38 and a second proximal leg 40. In some embodiments, the first proximal leg 38 and the second proximal leg 40 extend downwardly from a central portion 42 of the retainer 18. In some embodiments, the retainer 18 may include a first distal leg 44 and a second distal leg 46. In some embodiments, the first distal leg 44 and the second distal leg 46 may extend downwardly from the central portion 42. In some embodiments, the first proximal leg 38 and the first distal leg 44 may be on an opposite side of the midline 29 as the second proximal leg 40 and the second distal leg 46. In some embodiments, the first distal leg 44 and the second distal leg 46 may extend from the nose portion 34.

In some embodiments, the first retaining edge 30 may extend between the first proximal leg 38 and the first distal leg 44. In some embodiments, the second retaining edge 32 may extend between the second proximal leg 40 and the second distal leg 46. In some embodiments, the first proximal leg 38 and the first distal leg 44 may be configured to inhibit longitudinal movement of the first wing 26. In some embodiments, the first proximal leg 38 and the first distal leg 44 may contact the first wing 26. In some embodiments, the second proximal leg 40 and the second distal leg 46 may be configured to inhibit longitudinal movement of the second wing 28. In some embodiments, the second proximal leg 40 and the second distal leg 46 may contact the second wing 28.

In some embodiments, one or more of the first proximal leg 38, the second proximal leg 40, the first distal leg 44, and the second distal leg 46 may include a footing that may be laterally offset from a central or longitudinal axis of the retainer 18 and coupled to the one or more anchor pads 12. In some embodiments, the footings may support the retainer 18 on the one or more anchor pads 12. The footings may be further described in U.S. Pat. No. 11,020,565, filed Jul. 30, 2010, entitled “SECUREMENT DEVICE,” which is hereby incorporated by reference in its entirety.

In some embodiments, the securement device 10 may include an extension tube securement element 48 disposed on the upper surface of the one or more anchor pads 12. In some embodiments, the extension tube securement element 48 may include a clip, a fastener, or another suitable element that may be used to couple the extension tube 25 to the one or more anchor pads 12. As illustrated in FIG. 1A, for example, the extension tube securement element 48 may include a clip having opposing flexible arms configured to flex away from each other during pressing of the extension tube 25 into the clip and to come back towards each other to secure the extension tube 25 within the clip in a press fit. In some embodiments, the extension tube 25 may be formed into a U-shape between the side port and the clip such that the extension tube 25 stays close to the patient and does not catch on anything that could disturb a position of the catheter assembly 20. In some embodiments, the extension tube 25 may extend through a passage 49 proximate a downwardly extending portion of the retainer 18, as illustrated, for example, in FIG. 1H.

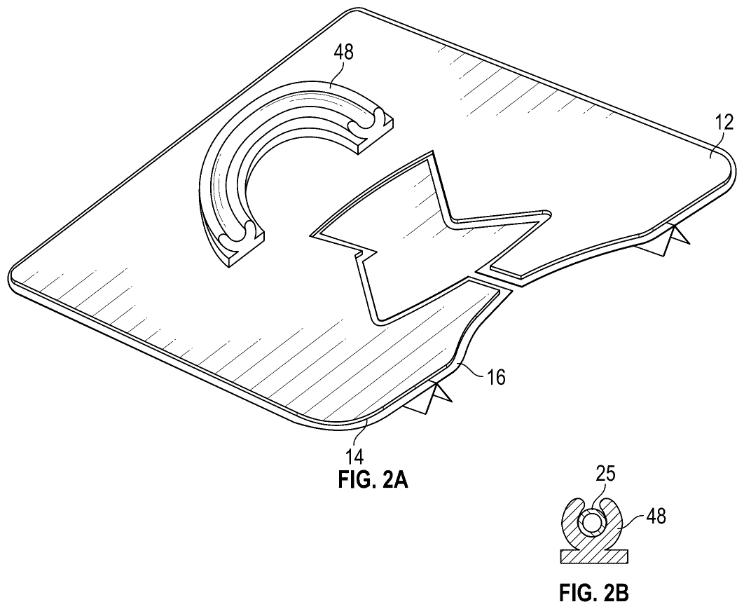

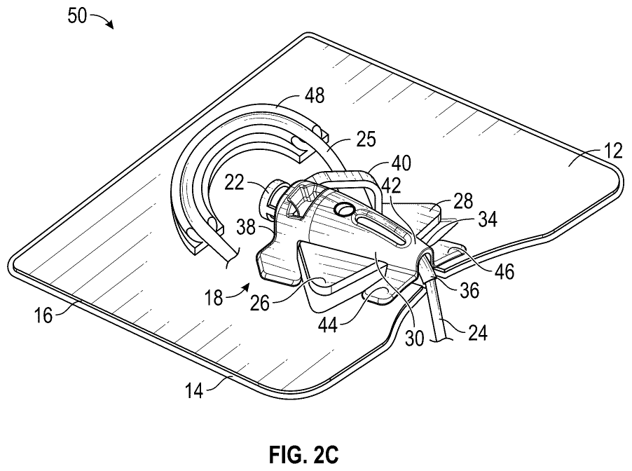

Referring now to FIGS. 2A-2C, a securement device 50 is illustrated, according to some embodiments. In some embodiments, the securement device 50 may be similar or identical to the securement device 10 of FIGS. 1A-1I in terms of one or more features and/or operation. In some embodiments, the extension tube securement element 48 may include a track, and the extension tube 25 of the catheter assembly 20 may be configured to be press fit within the track. In some embodiments, the track may be U-shaped and configured to turn the extension tube 25 180 degrees. In some embodiments, the track may include opposing flexible arms configured to flex away from each other during pressing of the extension tube 25 into the track and to come back towards each other to secure the extension tube 25 within the track in a press fit. In some embodiments, the track may facilitate the extension tube 25 staying close to the patient and not catching on anything that could disturb a position of the catheter assembly 20.

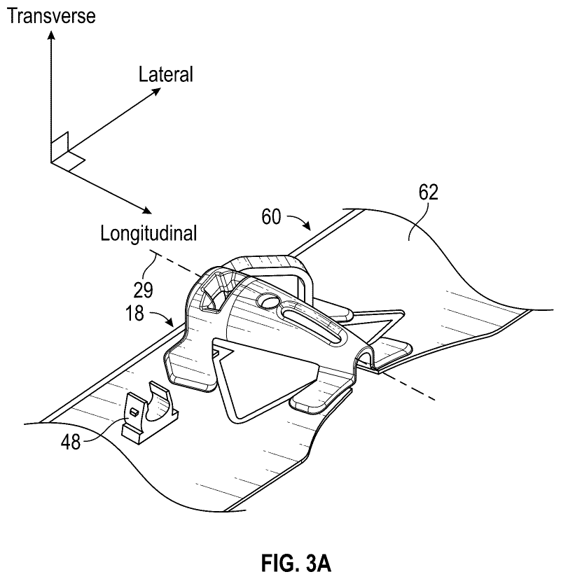

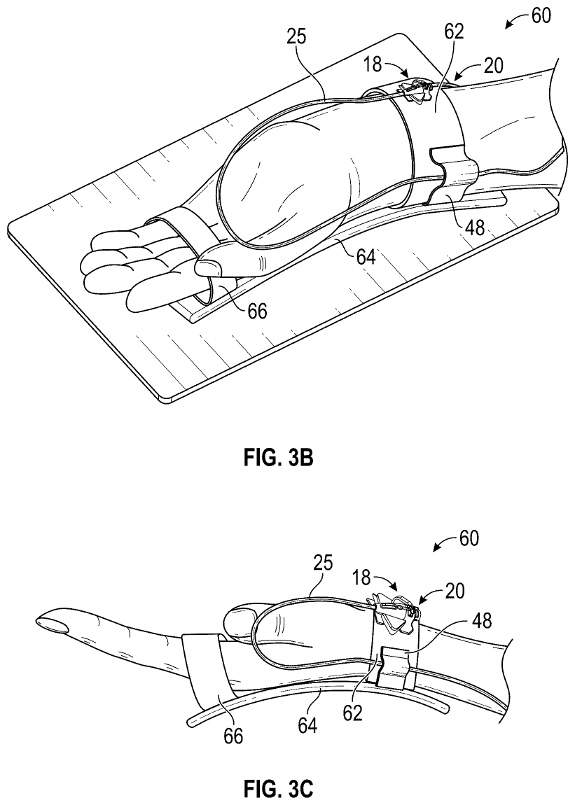

Referring now to FIGS. 3A-3C, a securement device 60 for securing a catheter assembly may include a bracelet 62 configured to wrap around a wrist of a patient. In some embodiments, the catheter assembly may include the catheter assembly 20 described with respect to FIGS. 1-2. In some embodiments, the securement device 60 may be similar or identical to the securement device 10 of FIGS. 1A-1I and/or the securement device 50 of FIGS. 2A-2C in terms of one or more features and/or operation.

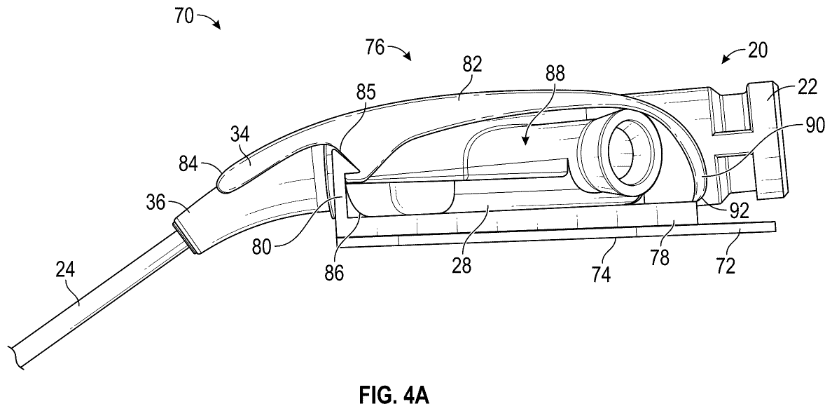

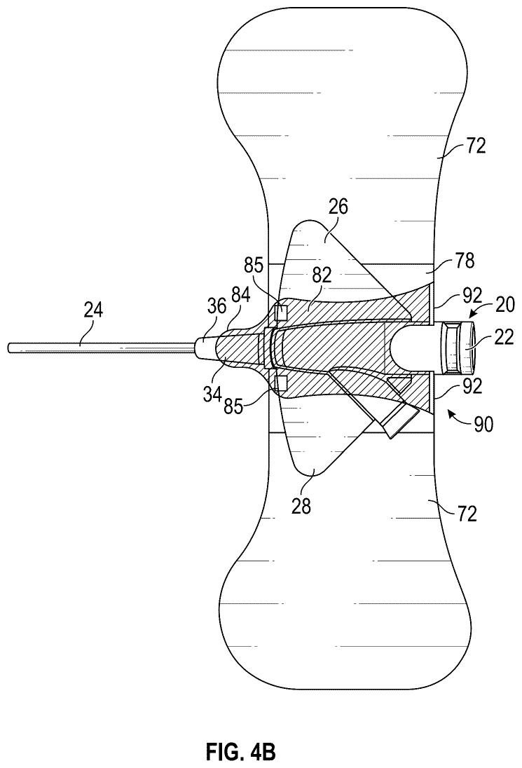

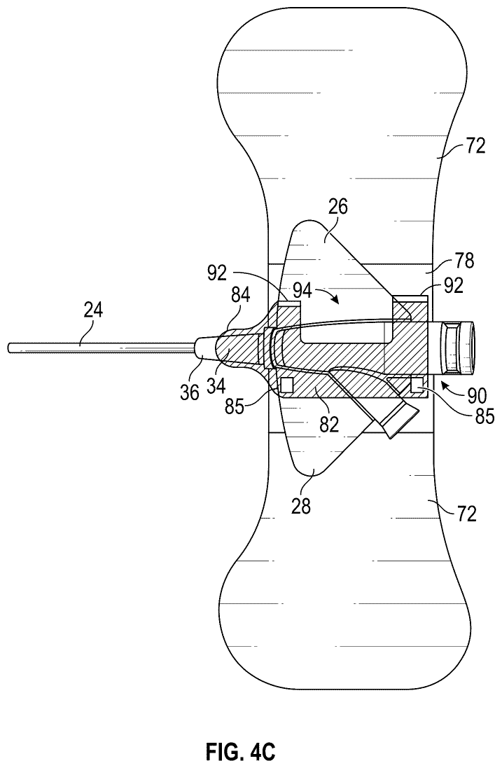

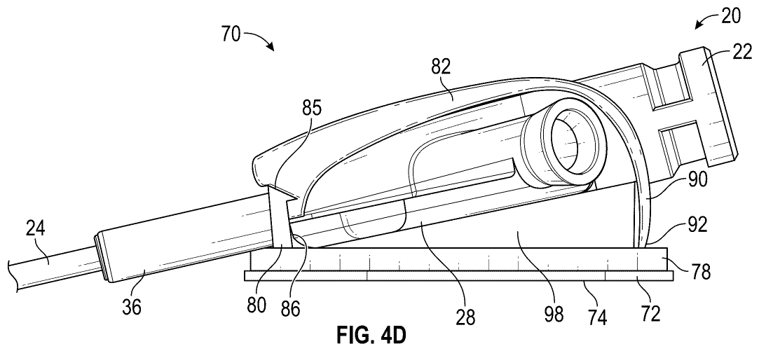

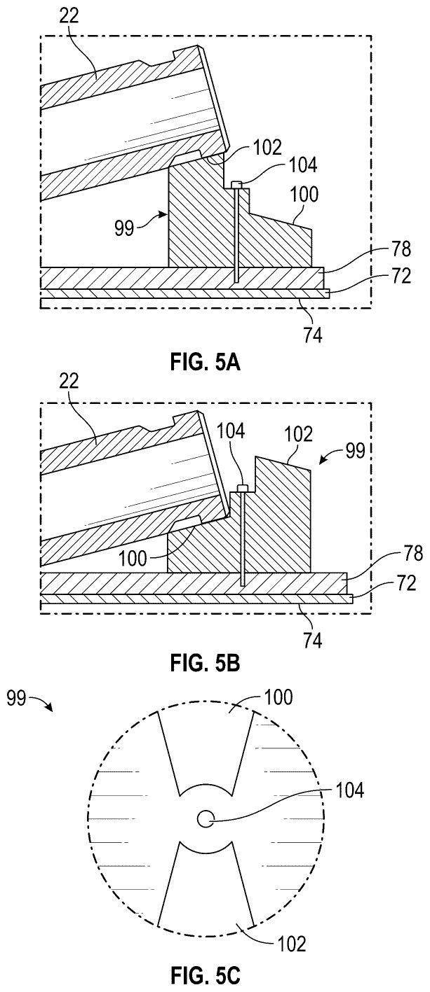

In some embodiments, the bracelet 62 may include an elastic band. In some embodiments, an interior of the bracelet 62 configured to contact skin of the patient may include rubber to avoid slipping on the skin. As illustrated in FIG. 3, in some embodiments, the securement device 60 may include the retainer 18 of FIGS. 1-2 positioned on an upper surface of the bracelet 62. Alternatively, in some embodiments, the securement device 60 may include another retainer, such as, for example, the retainer 76 of FIG. 4 or 5.

As illustrated in FIG. 3A, for example, the extension tube securement element 48 may include a clip having opposing flexible arms configured to flex away from each other during pressing of the extension tube 25 into the clip and to come back towards each other to secure the extension tube 25 within the clip in a press fit. Alternatively, the extension tube securement element 48 may include a barrette-like mechanism as illustrated in FIGS. 3B-3C. In some embodiments, the extension tube securement element 48 may include another suitable element to couple the extension tube 25 to the bracelet 62.

In some embodiments, the securement device 60 may include the extension tube securement element 48 disposed on the upper surface of the bracelet 62. In some embodiments, the securement device 60 may include a rigid support 64. In some embodiments, the rigid support 64 may include a first end coupled to the bracelet 62. In some embodiments, the rigid support 62 may extend from the bracelet 62 toward fingers of the patient to support the fingers in an extended position.

In some embodiments, the securement device 60 may include a band 66 coupled to a second end of the rigid support 64. In some embodiments, the band 66 may be configured to wrap around the fingers. In some embodiments, the band 66 may include an elastic band. In some embodiments, the band 66 may include a strip that wraps around the fingers. In some embodiments, an end of the strip may be secured to another portion of the strip via VELCRO® or another suitable fastening means. In some embodiments, the strip may facilitate securement of the band 66 to the fingers without disturbing an insertion site of the catheter assembly.

Referring now to FIG. 4A-4D, a securement device 70 may be similar or identical to the securement device 10 of FIGS. 1A-1I, the securement device 50 of FIGS. 2A-2C, and/or the securement device 60 of FIGS. 3A-3C in terms of one or more features and/or operation. In some embodiments, the securement device 70 may include one or more anchor pads 72, which may include an adhesive surface 74 disposed on an underside thereof and that is configured to attach to the skin surface of a patient. In some embodiments, the securement device 70 may include a retainer 76, which may include a bottom cover 78 fixed with respect to an upper surface of the one or more anchor pads 72. In some embodiments, the bottom cover 78 may include a latch feature 80. In some embodiments, the latch feature 80 may include an upwardly extending arm and/or a protruding tooth.

In some embodiments, the securement device 70 may include a top cover 82 hingedly coupled to an upper surface of the bottom cover 78. In some embodiments, the top cover 82 may include a distal end 84, which may include the nose portion 34 configured to deflect the strain relief of the catheter assembly 20 at a predetermined angle. In some embodiments, the top cover 82 may include a corresponding latch feature 85, which may include a recess sized and configured to receive the tooth of the latch feature 80. In some embodiments, in response to the top cover 82 moving from an open position to a closed position, the latch feature 80 and the corresponding latch feature 85 may be secured together. In some embodiments, when the top cover 82 is in the closed position, the retainer 76 may be configured to contact the catheter assembly 20 and secure the catheter assembly 20 between the top cover 82 and the bottom cover 78. In some embodiments, the upwardly extending arm of the latch feature 80 may be configured to flex to allow movement of the top cover 82 from the closed position to the open position.

In some embodiments, the top cover 82 may be configured to contact an upper surface of a particular wing of the catheter assembly 20, such as the first wing 26, when the top cover 82 is in the closed position to inhibit transverse movement of the catheter assembly 20. In some embodiments, the latch feature 80 may be configured to contact a distalmost surface 86 of the particular wing when the top cover 82 is in the closed position to inhibit distal movement of the catheter assembly 20. In some embodiments, the top cover 82 may include a gap 88 disposed between the corresponding latch feature 85 and the proximal end to receive the side port of the catheter adapter 22 and at least a portion of the particular wing of the catheter assembly 20.

As illustrated in FIG. 4B, in some embodiments, the top cover 82 may include a proximal end 90 hingedly coupled to the upper surface of the bottom cover 78. In some embodiments, the proximal end 90 may include one or more hinges 92 coupled to the upper surface of the bottom cover 78. In some embodiments, the hinges 92 may be living hinges or other suitable hinges. In some embodiments, the corresponding latch feature 85 may be disposed between the proximal end 90 and the distal end 84. In some embodiments, the top cover 82 may be aligned with the midline 29 in response to the top cover 82 being in the open position and the closed position.

In some embodiments, the retainer 76 may include a shape similar to the retainer 18 of FIG. 1, for example, and the first proximal leg 38 and the second proximal leg 40 may each include a hinge 92. In some embodiments, the bottom cover 78 may include multiple latch features 80. In some embodiments, the distal end 84 of the top cover 82 may include multiple corresponding latch features 85 configured to couple with multiple latch features 80 in the closed position.

As illustrated in FIG. 4C, in some embodiments, the top cover 82 may include a side 94, such as a right side or a left side, which may include the hinges 92 coupled to the upper surface of the bottom cover 78. In some embodiments, the corresponding latch feature 85 may be disposed on an opposite side of the midline 29 than the hinges 92. In some embodiments, the hinges 92 on the side 94 of the top cover 82 may include a distal hinge and a proximal hinge. In these embodiments, the bottom cover 78 may include multiple latch features 80. In some embodiments, an opposite side 96 as the side 94 of the top cover 82 may include multiple corresponding latch features 85 configured to couple with the multiple latch features 80 in the closed position.

As illustrated in FIG. 4D, in some embodiments, the bottom cover 78 may include or be coupled to a wedge-shaped platform 98 configured to support the catheter assembly 20 at a predetermined angle. For example, an upper surface of the wedge-shaped platform 98 may be angled at 30 to 45 degrees, inclusive, with respect to the bottom cover 78 or the adhesive surface 74.

Referring now to FIGS. 5A-5C, in some embodiments, the bottom cover 78 may be coupled to a rotatable wedge 99. In some embodiments, the rotatable wedge 99 may include a first platform 100 at a first height and a second platform 102 at a second height greater than the first height. In some embodiments, a retainer, such as the retainer 70 of FIGS. 4A-4D, for example, may include a pin 104 extending through the rotatable wedge 99 to the bottom cover 78, and the rotatable wedge 99 may rotate around the pin 104. In some embodiments, the first platform 100 and the second platform 102 may be angled downwardly away from the pin 104 and towards an outer edge of the rotatable wedge 99, which may allow each of the first platform 100 and the second platform 102 to support the catheter assembly 20 at a predetermined angle.

In some embodiments, the top cover 82 and/or the bottom cover 78 may be constructed of plastic or another suitable rigid or semi-rigid material to facilitate securement of the catheter assembly 20. In some embodiments, the bottom cover 78 may be more rigid than the anchor pad 72, which may facilitate securement of the pin 104 within the bottom cover 78.

All examples and conditional language recited the present disclosure are intended for pedagogical objects to aid the reader in understanding the present disclosure and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the present disclosure.

What is claimed:

1. A securement device for securing a catheter assembly, the securement device comprising:
one or more anchor pads comprising an adhesive surface disposed on an underside thereof and that is configured to attach to a skin surface of a patient;
a retainer positioned on an upper surface of the one or more anchor pads, wherein the retainer comprises:
a first retaining edge configured to contact a top of a first wing of the catheter assembly;
a second retaining edge configured to contact a top of a second wing of the catheter assembly opposite the first wing; and
a catheter angle adjuster a nose portion configured to deflect a strain relief of the catheter assembly at a predetermined angle.

2. The securement device of claim 1, wherein the retainer further comprises:
a first proximal leg and a second proximal leg, wherein the first proximal leg and the second proximal leg extend downward from a central portion; and
a first distal leg and a second distal leg, wherein the first distal leg and the second distal leg extend downward from the central portion, wherein the first proximal leg and the first distal leg are on an opposite side of a midline as the second proximal leg and the second distal leg,
wherein the first retaining edge extends between the first proximal leg and the first distal leg, wherein the second retaining edge extends between the second proximal leg and the second distal leg, wherein the first proximal leg and the first distal leg are configured to inhibit longitudinal movement of the first wing, wherein the second proximal leg and the second distal leg are configured to inhibit longitudinal movement of the second wing.

3. The securement device of claim 1, further comprising an extension tube securement element disposed on the upper surface of the one or more anchor pads.

4. The securement device of claim 3, wherein the extension tube securement element comprises a track, wherein an extension tube of the catheter assembly is configured to be press fit within the track.

5. The securement device of claim 4, wherein the track is U-shaped and configured to turn the extension tube 180 degrees.

6. The securement device of claim 1, wherein the one or more anchor pads comprises a first anchor pad and a second anchor pad laterally offset from a midline, wherein the retainer is positioned on an upper surface of each of the first anchor pad and the second anchor pad, wherein the first anchor pad and the second anchor pad are shaped so as to allow ingress and egress of a portion of the catheter assembly therebetween.

7. The securement device of claim 1, wherein the catheter angle adjuster comprises the nose portion configured to deflect the strain relief of the catheter assembly at the predetermined angle.

8. A securement device for securing a catheter assembly, the securement device comprising:
a bracelet configured to wrap around a wrist of a patient;
a retainer positioned on an upper surface of the bracelet, wherein the retainer comprises:
a first retaining edge configured to contact a top of a first wing of the catheter assembly;
a second retaining edge configured to contact a top of a second wing of the catheter assembly opposite the first wing; and
a nose portion configured to deflect a strain relief of the catheter assembly at a predetermined angle; and
an extension tube securement element disposed on the upper surface of the bracelet.

9. The securement device of claim 8, further comprising a rigid support, wherein the rigid support comprises a first end coupled to the bracelet and configured to extend from the bracelet toward fingers of the patient.

10. The securement device of claim 9, further comprising a band coupled to a second end of the rigid support, wherein the band is configured to wrap around the fingers.

11. A securement device for securing a catheter assembly, the securement device comprising:
one or more anchor pads comprising an adhesive surface disposed on an underside thereof and that is configured to attach to a skin surface of a patient;
a retainer, comprising:
a bottom cover fixed with respect to an upper surface of the one or more anchor pads, wherein the bottom cover comprises a latch feature;
a top cover hingedly coupled to an upper surface of the bottom cover,
wherein the top cover comprises:
a distal end comprising a nose portion configured to deflect a strain relief of the catheter assembly at a predetermined angle; and
a corresponding latch feature, wherein in response to the top cover moving from an open position to a closed position, the latch feature and corresponding latch feature are secured together, wherein when the top cover is in the closed position, the retainer is configured to secure the catheter assembly between the top cover and the bottom cover.

12. The securement device of claim 11, wherein the top cover further comprises:
a proximal end hingedly coupled to the upper surface of the bottom cover;
wherein the corresponding latch feature is disposed between the proximal end and the distal end, wherein the top cover is aligned with a midline in response to the top cover being in the open position and the closed position.

13. The securement device of claim 12, wherein the proximal end comprises a plurality of hinges coupled to the upper surface of the bottom cover, wherein the bottom cover comprises a plurality of latch features, wherein the distal end comprises a plurality of corresponding latch features configured to couple with the plurality of latch features in the closed position.

14. The securement device of claim 11, wherein the top cover further comprises a side comprising a hinge coupled to the upper surface of the bottom cover, wherein the corresponding latch feature is disposed on an opposite side of a midline than the hinge.

15. The securement device of claim 14, wherein the side comprises a distal hinge and a proximal hinge, wherein the bottom cover comprises a plurality of latch features, wherein an opposite side as the side comprises a plurality of corresponding latch features configured to couple with the plurality of latch features in the closed position.

16. The securement device of claim 11, wherein the top cover is configured to contact an upper surface of a wing of the catheter assembly when the top cover is in the closed position to inhibit transverse movement of the catheter assembly.

17. The securement device of claim 11, wherein the latch feature is configured to contact a distalmost surface of a wing of the catheter assembly when the top cover is in the closed position to inhibit distal movement of the catheter assembly.

18. The securement device of claim 11, wherein the top cover comprises a gap disposed between the corresponding latch feature and a proximal end of the top cover to receive a side port of a catheter adapter of the catheter assembly and at least a portion of a wing of the catheter assembly.

19. The securement device of claim 11, wherein the bottom cover is coupled to a wedge-shaped platform configured to support the catheter assembly at a predetermined angle.

20. The securement device of claim 11, wherein the bottom cover is coupled to a rotatable wedge, wherein the rotatable wedge comprises a first platform at a first height and a second platform at a second height greater than the first height, wherein the retainer further comprises a pin extending through the rotatable wedge to the bottom cover.
